(19) United States
(12) Patent Application Publication
(10) Pub. No.: US 2025/0095771 A1
(43) Pub. Date: Mar. 20, 2025

(54) METHODS OF VACCINE DESIGN

(71) Applicant: NEC Laboratories Europe GmbH, Heidelberg (DE)

(72) Inventors: Filippo GRAZIOLI, Heidelberg (DE); Anja MOESCH, Heidelberg (DE); Brandon MALONE, Heidelberg (DE)

(21) Appl. No.: 18/729,517

(22) PCT Filed: Jan. 18, 2022
(86) PCT No.: PCT/EP2022/051042

§ 371 Date: Jul. 17, 2024

(51) Int. Cl. G16B 5/20; G16B 15/30; G16B 20/20

(52) U.S. Cl. CPC G16B 5/20; G16B 15/30; G16B 20/20

(57) ABSTRACT

A method for selecting an amino acid sequence for inclusion in a neoantigen vaccine from a set of candidate neoantigen amino acid sequences is provided. A plurality of cancer cells are simulated based on a set of input data related to a patient by predicting a cell surface presentation of each cancer cell. For each candidate neoantigen amino acid sequence, a likelihood is predicted of each candidate neoantigen amino acid sequence eliciting an immune response to the plurality of cancer cells based on the predicted cell surface presentation of each cancer cell. One or more amino acid sequences is selected for inclusion in the neoantigen vaccine that maximizes a likelihood of the neoantigen vaccine eliciting an immune response to the plurality of cancer cells based on the predicted likelihood of each candidate neoantigen amino acid sequence eliciting an immune response to the plurality of cancer cells.

This application is a U.S. National Phase application under 35 U.S.C. § 371 of International Application No. PCT/EP2022/051042, filed on Jan. 18, 2022. The International Application was published in English on Jul. 27, 2023 as WO 2023/138755 A1 under PCT Article 21 (2).

### FIELD

The present invention relates to the field of vaccine design and creation, including the selection of amino acid sequences for inclusion in a vaccine and the synthesis of one or more amino acid sequences to create a vaccine.

### BACKGROUND

In recent years, there has been increasing interest in the development of therapeutic cancer vaccines, a form of cancer immunotherapy which is used to stimulate a patient's immune system to attack and kill cancer cells. Healthy cells do not contain the same DNA changes which are present in cancer cells, which makes these DNA changes, along with the associated proteins and peptides synthesised and processed in the cancer cells, a possible target for a vaccine.

Neoantigen vaccines, in particular, aim to enable the human immune system to target neoantigens, which are proteins that form on cancer cells in response to mutations in the DNA of a tumour, while also avoiding off-target or auto-immune responses.

Inside all human cells, the DNA is transcribed into messenger RNA (mRNA) and then the mRNA is translated into proteins. If a cell's DNA contains a mutation (i.e. a change in the DNA), this will be also transcribed into the mRNA, and can cause changes in the amino acid sequence of proteins synthesised within the cell. These altered proteins are typically not useful to the cell and are therefore processed in one of two antigen-processing pathways, each of which always leads to cleaving the protein into peptides.

Endogenous processing pathway: under this pathway, within the same cell in which the protein was synthesized, the proteasome splits the protein into sub-units called peptides. These peptides can then be transported into the endoplasmic reticulum (ER), where they can bind to the major histocompatibility complex I protein (MHC-I). After having bound to an MHC-I protein, the peptide-MHC-I complex may then be presented on the surface of the cell. Once the peptide-MHC-I complex is presented on the surface of the antigen-presenting cell (APC), a T cell can bind to it with its T cell receptor (TCR), which recognizes the peptide, then also referred to as epitope, in combination with its co-receptor, the cluster of differentiation 8 receptor (CD8+). The T cell will induce cell death of the presenting cell. For this reason, the CD8+ T cells are also called cytotoxic T cells (CTLs).

Exogenous processing pathway: under this pathway, a protein containing a mutation is absorbed by a cell through endocytosis. Analogously to what happens in the endogenous processing pathway, the malformed protein is degraded into small sequences of amino acids (peptides) by the proteases. The peptides then bind to major histocompatibility complex II proteins (MHC-II), and the peptide-MHC-II complex is presented on the cell surface of an antigen presenting cell (APC). T cells with the cluster of differentiation 4 receptor protein (CD4+) bind to the peptide-MHC-II complex. Following this event, CD4+ T cells release substances called cytokines which can activate B cells or CTLs. Due to this, CD4+ T cells are also called helper T cells.

In humans, the MHC is also referred to as human leukocyte antigen (HLA). In the human genome, there are MHC-I (also referred to as HLA-I) and MHC-II (also referred to as HLA-II) genes. Each individual has a set of three major HLA-I genes: HLA-A, HLA-B, and HLA-C. For each of these genes, a person has two versions, called alleles, which are inherited by the father and by the mother. Hence, in the body of an individual, there can be up to six different major HLA-I molecules, which each bind to a different set of epitopes.

For HLA-II, there are also three major genes: HLA-DR, HLA-DP and HLA-DQ. For each gene, each person has two alleles, inherited by the father and the mother. The HLA-II system, however, is more complex than the HLA-I system: the HLA-II molecules are heterodimer complexes formed by polymorphic genes (alpha and beta chains). Due to this, each person has up to 12 HLA-II complexes. Additionally, HLA-II presented epitopes are longer and vary more in length compared to HLA-I.

The endogenous and exogenous processing pathways are discussed in more detail in Alberts, B.; Johnson, A.; Lewis, J.; Raff, M.; Roberts, K. & Walter, P. Molecular Biology of the Cell. Garland Science, 2002.

As described above, the pathways through which epitopes, including neoepitopes/neoantigens, can elicit an immune response are complex and include many steps. Any of these steps (e.g. the binding of an epitope with an HLA molecule, or the presentation of the epitope-HLA on the surface of the cell) could fail. Due to this, certain tumour mutations can be good candidates for neoantigen vaccines, while others can be less promising. For example, some mutations might never be translated into protein, in which case the pathways described above are never activated in the first place. Other mutations, which are translated into proteins, can result into peptides which do not bind well with the HLA complexes of a given individual. Furthermore, even if a neoepitope-MHC complex is presented on the surface of a cell, it might be possible that T cells do not recognize it.

In order to develop effective neoantigen vaccines, it is therefore important to understand which neoantigen candidates are the best to include in a vaccine.

### SUMMARY

In an embodiment, the present disclosure provides a computer-implemented method of selecting one or more amino acid sequences for inclusion in a neoantigen vaccine from a set of candidate neoantigen amino acid sequences, the method comprising: retrieving a set of input data related to a patient; simulating a plurality of cancer cells based on the set of input data, wherein simulating each cancer cell of the plurality of cancer cells comprises predicting a cell surface presentation of said cancer cell of the plurality of cancer cells; for each candidate neoantigen amino acid sequence of the set of candidate neoantigen amino acid sequences, predicting a likelihood of the candidate neoantigen amino acid sequence eliciting an immune response to the plurality of cancer cells based on the predicted cell surface presentation of each cancer cell; and selecting one or more amino acid sequences of the set of candidate neoantigen amino acid sequences for inclusion in the neoantigen vaccine that maximizes a likelihood of the neoantigen vaccine eliciting an immune response to the plurality of cancer cells based on the predicted likelihood of each candidate neoantigen amino acid sequence eliciting an immune response to the plurality of cancer cells.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a specific example of a process used to select neoantigen candidate amino acid sequences for a neoantigen vaccine;

FIG. 2 illustrates a network flow problem to be solved to select an optimal neoantigen vaccine composition in an embodiment of the invention; and

FIG. 3 illustrates a network flow problem to be solved to select an optimal neoantigen vaccine composition in another embodiment of the invention.

### DETAILED DESCRIPTION

Aspects of the invention provide a method and a system for selecting a set of candidate neoantigen elements for inclusion in a vaccine such that a likelihood of the vaccine eliciting an immune response to the cancer cells of a patient is maximised.

According to a first aspect of the invention, a computer-implemented method of selecting one or more amino acid sequences for inclusion in a neoantigen vaccine from a set of candidate neoantigen amino acid sequences is provided. The method comprises: retrieving a set of input data related to a patient; simulating a plurality of cancer cells based on the set of input data, wherein simulating each cancer cell comprises predicting the cell surface presentation of said cancer cell; for each candidate neoantigen amino acid sequence, predicting a likelihood of said candidate neoantigen amino acid sequence eliciting an immune response to the cancer cells based on the predicted cell surface presentation of each cancer cell; and selecting the one or more amino acid sequences for inclusion in the vaccine that maximise a likelihood of the vaccine eliciting an immune response to the cancer cells based on the predicted likelihood of each candidate neoantigen amino acid sequence eliciting an immune response to the cancer cells.

The first aspect of the invention allows for the composition of a therapeutic cancer vaccine to be optimised. In contrast to conventional approaches, the present invention does this by simulating the population of cancer cells in a patient and then predicting a likelihood of a vaccine eliciting an immune response to those cancer cells. By maximising this likelihood, a set of vaccine elements (amino acid sequences) may then be selected so as to optimise the composition of the vaccine.

A further advantage of the present invention is that it allows for an evaluation of the immune response likely to be induced by a vaccine along with an estimation of the quantity of cancer cells killed. This allows a margin of vaccine efficacy to be estimated in a way that is not possible with conventional approaches to selecting the composition of a vaccine.

The skilled person will of course understand that maximising a likelihood of the vaccine eliciting an immune response to the cancer cells will involve using one of a number of optimisation process, each of which may lead to different maxima being reached. Likewise, there may be different measures of the likelihood of the vaccine eliciting an immune response to the cancer cells. As such, this step can be implemented in different ways, which may lead to different amino acid sequences being selected for inclusion in the vaccine.

Advantageously, the step of simulating a plurality of cancer cells involves modelling one or more of the biochemical processes occurring within a cancer cell. To this end, the set of input data advantageously comprises one or more of: an indication of the patient's HLA-I alleles; gene expression information; a set of identified gene variants; binding affinity indicators for each tuple of candidate neoantigen amino acid sequence and HLA-I allele; and presentation indicators for each tuple of candidate neoantigen amino acid sequence and HLA-I allele.

The biochemical processes which may be simulated preferably include one or more of the steps of the endogenous processing pathway, namely transcription, translation, intracellular processing, HLA binding, and cell surface presentation.

In order to simulate the transcription step of the endogenous processing pathway, the step of simulating a plurality of cancer cells preferably comprises predicting the presence or absence of each of the identified gene variants in each of the plurality of cancer cells based on a statistical distribution of the identified gene variants.

In order to simulate the translation the step of the endogenous processing pathway, the step of simulating a plurality of cancer cells preferably comprises estimating the abundance of one or more proteins synthesized in each cancer cell based on the gene expression information and on the gene variants predicted to be present in each cancer cell.

In order to simulate the intracellular processing step of the endogenous processing pathway, the step of simulating a plurality of cancer cells comprises estimating the abundance of one or more peptides processed in each cancer cell based on the estimated abundance of one or more proteins synthesised in said cancer cell and on a likelihood of each of the one or more proteins being split into the one or more peptides.

In order to simulate the HLA binding step of the endogenous processing pathway, the step of simulating a plurality of cancer cells preferably comprises simulating the binding of the one or more peptides to HLA molecules to estimate a likelihood of one or more peptide-HLA complexes being present in each cancer cell, wherein simulating the binding of peptides to HLA molecules is based on the abundance of said one or more peptides and on the binding affinity indicators for each tuple of candidate neoantigen amino acid sequence and HLA-I allele.

In order to simulate the cell surface presentation step of the endogenous processing pathway, the step of simulating a plurality of cancer cells preferably comprises predicting the cell surface presentation of each cancer cell based on the likelihood of the one or more peptide-HLA complexes being present within each cancer cell and on the presentation indicators for each tuple of candidate neoantigen amino acid sequence and HLA-I allele.

Simulating a population of cancer cells (also referred to as cancer digital twins) through probabilistic simulations of the endogenous processing pathway allows statistical predictions (including machine learning predictions) to be combined with mechanistic models of the cancer cells. In other words, features of data-driven approaches are combined with features of model-driven approaches, thereby combining information extracted from data with knowledge of the biochemistry of cancer cells. This combined approach allows for cancer cells to be simulated more accurately.

Simulating the binding of peptides to HLA molecules, in particular, allows for improvements in the simulation of cancer cells. A given neoantigen and a given HLA-I molecule can bind with a given affinity, and pairs with a stronger affinity have a higher probability to bind. Pairs with lower affinity may also bind, but with a lower probability. However, at any given time the amount of neoantigens and HLA-I molecules present within a cancer cell is limited. The binding of neoantigens with HLA-I molecules is, therefore, competitive. By taking into account the estimated abundance of peptides within a cancer cell and the affinity of said peptides with HLA-I molecules, this competitive process may be modelled to estimate the likelihood of one or more peptide-HLA complexes being present in each cancer cell.

This process strongly influences the cell surface presentation of cancer cells and, as a result, the immune response to those cells. As such, simulating this process allows for cancer cells to be simulated more accurately.

The step of predicting an immune response advantageously comprises estimating a likelihood of a patient's immune system including T cells having receptors which bind with the surface of the cancer cell. The immune response could be predicted directly by predicting TCR-peptide-HLA binding, but it is preferable to estimate the likelihood of a cancer cell presenting a neoantigen to be killed by a T cell, i.e. how likely it will be that there is a T cell that is able to access the tumour and recognize the neoantigen. This can be calculated by taking information of the tumour infiltrating lymphocytes (TIL), i.e. T cells that are present in the tumour sequencing data, into account. This data consists of TCR information like V, D and J alleles, CDR3 sequences, TIL marker genes and corresponding cancer cell markers.

To this end, the input data preferably further includes TCR repertoire and relevant gene expression data when a likelihood of a patient's immune system include T cells having receptors which bind with the surface of the cancer cell is to be estimated. This data can then be used to determine the T cells present in a patient's immune system and to estimate the likelihood of any of these having receptors which bind with the surface of the cancer cell.

As noted above, there are various optimisation processes which could be used to maximise a likelihood of the vaccine eliciting an immune response to the cancer cells. In one such process, the step of selecting the one or more amino acid sequences for inclusion in the vaccine comprises applying a mathematical optimisation algorithm to minimise a likelihood of the vaccine eliciting no immune response to the cancer cells.

The skilled person will understand that maximising a likelihood of an event occurring is equivalent to minimising the likelihood of that event not occurring. Reframing the step of selecting one or more amino acid sequences as minimising a likelihood of the vaccine eliciting no immune response to the cancer cells advantageously allows for a mathematical optimisation algorithm to be used which is based on minimising the flow in a network where one set of nodes correspond to candidate neoantigen amino acid sequences, one set of nodes correspond to the plurality of cancer cells, and there is one sink. The optimised vaccine constituents therefore minimise the likelihood of no response across the whole population of cancer cells.

The variables of the mathematical optimisation algorithm preferably comprise: (a) a binary indicator variable for each candidate neoantigen amino acid sequence which indicates whether the candidate amino acid is included in a vaccine; and (b) a continuous variable for each cancer cell which gives a log likelihood of no immune response being elicited by a candidate neoantigen amino acid sequence to said cancer cell.

The continuous variable for each cancer cell which gives a log likelihood of no immune response being elicited by a candidate neoantigen amino acid sequence to said cancer cell can be estimated using the predicted likelihood of each candidate neoantigen amino acid sequence eliciting an immune response to the cancer cells, simplifying the optimisation problem to finding the binary indicator variables which minimise the total flow from the amino acid sequence nodes to the cancer cell nodes.

Alternatively, the step of selecting the one or more amino acid sequences for inclusion in the vaccine may comprise applying a mathematical optimisation algorithm to minimise the estimated likelihood of the vaccine eliciting no immune response to the cell for which the estimated likelihood of no immune response being elicited by the vaccine is highest.

In this approach, a mathematical optimisation algorithm is used which is also based on minimising the flow in a network where one set of nodes correspond to candidate neoantigen amino acid sequences and one set of nodes correspond to the plurality of cancer cells. In this case, however, each cell is a sink and the flow to each individual sink is minimised. The optimised vaccine constituents therefore minimise the likelihood of no immune response being elicited to any one cancer cell. Therefore, whereas the first mathematical optimisation algorithm results in a vaccine composition which kills the maximum number of cancer cells, this alternative mathematical optimisation algorithm results in a vaccine composition which maximises the likelihood of eliciting at least some immune response to all cancer cells.

The variables of this second mathematical optimisation algorithm preferably comprise: (a) a binary indicator variable for each candidate neoantigen amino acid sequence which indicates whether the candidate amino acid is included in a vaccine; and (b) a continuous variable for each cancer cell which gives a log likelihood of no immune response being elicited by a candidate neoantigen amino acid sequence to said cancer cell. These are the same variables as discussed above. The variables preferably further comprise: (c) a continuous variable for each cancer cell which gives a log likelihood of no immune response being elicited by a vaccine comprising a subset of the set of candidate neoantigen amino acid sequences; and (d) a continuous variable which gives a maximum log-likelihood that any one cancer cell does not respond to a vaccine comprising a subset of the set of candidate neoantigen amino acid sequences.

The continuous variable for each cancer cell which gives a log likelihood of no immune response being elicited by a vaccine comprising a subset of the set of candidate neoantigen amino acid sequences is used to calculate the continuous variable which gives a maximum log-likelihood that any one cancer cell does not respond to a vaccine comprising a subset of the set of candidate neoantigen amino acid sequences.

Whichever mathematical optimisation algorithm is used, it is preferable for this to be an integer linear program.

As will be understood, the vaccine platform used will constrain the total length of amino acid sequences included. As such, it is preferable for the method to further comprise assigning a cost to each candidate amino acid sequence, with the step of selecting the one or more amino acid sequences for inclusion in the vaccine constrained based on the cost assigned to each candidate amino acid sequence, such that the selected one or more amino acid sequences have a total cost below a predetermined threshold budget.

This constraint is most preferably used to constrain a mathematical optimisation algorithm used to select the one or more amino acid sequences for inclusion in the vaccine. Integer linear programs are especially well suited to solving such constrained optimisation problems.

According to a second aspect of the invention, a method of creating a vaccine is provided, the method comprising: selecting one or more amino acid sequences for inclusion in a vaccine from a set of predicted neoantigen candidate amino acid sequences by a method according to an embodiment of the first aspect of the invention; and synthesising the one or more amino acid sequences or encoding the one or more amino acid sequences into a corresponding DNA or RNA sequence and/or incorporating the DNA or RNA sequence into a genome of a bacterial or viral delivery system to create a vaccine.

According to a third aspect of the invention, a system for selecting one or more amino acid sequences for inclusion in a vaccine from a set of predicted neoantigen candidate amino acid sequences is provided, the system comprising at least one processor in communication with at least one memory device, the at least one memory device having stored thereon instructions for causing the at least one processor to perform a method according to an embodiment of the first aspect of the invention.

According to a fourth aspect of the invention, a computer readable medium is provided having computer executable instructions stored thereon for implementing a method according to an embodiment of the first aspect of the invention.

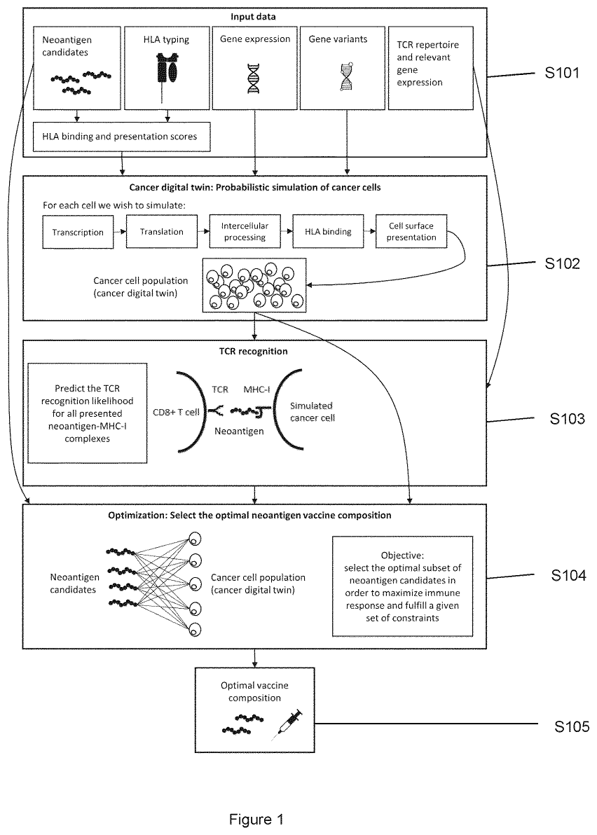

The following sets out a specific example of the selection of neoantigen candidate amino acid sequences for a neoantigen vaccine with reference to FIG. 1. In the proposed implementation set out below, note that any references indicated herein are incorporated by reference.

For each step discussed below that includes sampling from a distribution, these distributions can also be learned by a machine learning algorithm if appropriate data is available.

### Input Data

In step S101 a set of input data is retrieving which relates to a patient, and which advantageously includes an indication of the patient's HLA-I alleles; gene expression information; a set of identified gene variants; binding affinity indicators for each tuple of candidate neoantigen amino acid sequence and HLA-I allele; presentation indicators for each tuple of candidate neoantigen amino acid sequence and HLA-I allele; as well as TCR repertoire and relevant gene expression data. This data is related to a patient and may be used to simulate the cancer cells present in the patient.

A set of predicted candidate neoantigen amino acid sequences are also retrieved in step S101. The neoantigen candidates may be proteins which resemble the neoantigen proteins which form on cancer cells in response to the mutations in the DNA of a tumour so as to enable the creation of a protein-based vaccine or may comprise a DNA or RNA sequence so as to enable the creation of a DNA- or RNA-based vaccine, such as an mRNA vaccine.

Therefore, although the expression “neoantigen candidate amino acid sequence” refers to the sequence of amino acids which form a neoantigen protein, the vaccine itself need not comprise the selected amino acid sequences but rather may comprise a corresponding DNA or RNA sequence.

The candidate neoantigen amino acid sequences are referred to as “candidates” because they could, in principle, be selected as vaccine elements. However, a given neoantigen will typically not present on all possible cancer cells. Furthermore, many neoantigens will not elicit an immune response. The goal of the present invention is therefore to identify the optimal subset of the neoantigen candidates which maximizes the likelihood of having immune response.

The neoantigen candidates may also be used in step S102 to simulate cancer cells. This simulation could be carried out in a number of ways, but preferably involves simulating the steps of transcription, translation, intracellular processing, HLA binding, and cell surface presentation, so as to simulate a population of cancer cells, also referred to as cancer digital twins.

### Cancer Digital Twin: Probabilistic Simulation of Cancer Cells

In the first step of transcription, the presence of one or more gene variants in each simulated cancer cell is determined by sampling from a statistical distribution (e.g. a Bernoulli distribution). The population of simulated cancer cells are then assigned the gene variants according to this sampling.

As noted above, the input data includes a set of identified gene variants, and these are preferably the somatic variants, which can be derived from whole genome sequencing data. The identification of the somatic variants is well understood in the art and involves comparing the exome data for a tumour sample and with the exome data for healthy tissue, so as to identify gene variants which appear in the tumour sample but not in the healthy tissue. For example, the GATK Best Practices (https://software.broadinstitute.org/gatk/best-practices/workflow?id=11146 13.10.2021) could be used to implement this step.

A parametric distribution of gene variants may then be obtained by using the DNA variant allele frequency (VAF), which is the percentage of reads matched to the variant divided by the sum of all reads matched to the gene.

The translation step then aims to estimate the protein abundance in each simulated cancer cell from gene expression information. This involves sampling from a distribution of FPKM (fragments per kilobase per million mapped reads) values and FPKM variance values (based on 95% confidence interval) and multiplying this value by the RNA VAF, since the FPKM values are calculated based on all reads matching the gene but only a fraction of them (=VAF) contain the actual mutation.

To this end, the gene expression information retrieved in step S101 advantageously comprises a table of FPKM RNA-sequencing based gene expression data. Each gene is identified by its name and ENSG-identifier and has an FPMK value and an FPKM variance value. This input type may, for example be based on Cufflinks (http://cole-trapnell-lab.github.io/cufflinks/cufflinks/#fpkm-tracking-format 14.10.2021), which provides lower and higher bound of the 95% confidence interval, which can be used to calculate FPKM variance values.

Having estimated the protein abundance in each simulated cancer cell, the intracellular processing of each simulated cancer cell is simulated to estimate the abundance of peptides within each simulated cancer cell.

In order to aid this steps of simulating the cancer cell population, step S101 includes a step of receiving binding affinity indicators for each tuple of candidate neoantigen amino acid sequence and HLA-I allele and presentation indicators for each tuple of candidate neoantigen amino acid sequence and HLA-I allele, although step S101 may instead include a step of deriving binding affinity indicators for each tuple of candidate neoantigen amino acid sequence and HLA-I allele and presentation indicators for each tuple of candidate neoantigen amino acid sequence and HLA-I allele.

These indicators, also referred to as HLA binding and presentation scores, can be predicted by machine learning models, e.g. NetMHC (Gapped sequence alignment using artificial neural networks: application to the MHC class I system; Andreatta M, Nielsen M; Bioinformatics 32.4 (2016): 511-517) or MHCflurry (MHCflurry 2.0: Improved Pan-Allele Prediction of MHC Class I-Presented Peptides by Incorporating Antigen Processing; Timothy J. O'Donnell, Alex Rubinsteyn, Uri Laserson; Cell systems 11.1 (2020): 42-48) for MHC binding, as well as other predictors for other steps of intracellular processing to obtain a presentation score.

In addition to being based on the neoantigen candidates, these indicators are predicted based on an indication of the patient's HLA-I alleles, also referred to as HLA typing. This can be determined from WXS (whole exome sequencing) data of healthy cells. The HLA typing can either be at a gene level (HLA-A, HLA-B, HLA-C, etc.) or allele-specific, if available.

The intracellular processing step itself then simulates the peptide processing and assigns a weight to each peptide, which reflects the likelihood that the peptide will be present after the protein to which it belongs is processed. The weight is calculated from the presentation score but can also be implemented by using individual weights for each relevant processing step like cleavage, trimming, transport to the endoplasmic reticulum, etc.

Once the abundance of peptides in each simulated cancer cell is estimated, an HLA binding step is carried out to simulate the competitive binding of peptides to the available MHC (HLA) molecules by taking the MHC-peptide binding prediction score as well as the MHC molecules (derived from the HLA typing) into account.

Finally, the cell surface presentation of each cancer cell is simulated. The presentation score is used as a sampling probability to determine whether a peptide-HLA (also referred to as a peptide-MHC) complex present within each simulated cancer cell is also presented on the cell surface. In other words, the joint likelihood of peptide-HLA complex being present within each simulated cancer cell and of each peptide-HLA complex presenting on the surface of a cancer cell is used to simulate the cell surface presentation of each simulated cancer cell.

Having simulated the cell surface presentation of the population of simulated cancer cells, the TCR recognition likelihood for all presented neoantigen-MHC-I complexes is predicted in step S103.

### TCR Recognition

Rather than directly predicting TCR-peptide-HLA binding, it is preferable to estimate the likelihood of a cancer cell presenting a neoantigen to be killed by a T cell, i.e. how likely it will be that there is a T cell that is able to access the tumour and recognize the neoantigen. This can be calculated by taking information of the tumour infiltrating lymphocytes (TIL), i.e. T cells that are present in the tumour sequencing data, into account. This data consists of TCR information like V, D and J alleles, CDR3 sequences, TIL marker genes and corresponding cancer cell markers. This is advantageous as existing algorithms for predicting TCR-peptide-HLA binding are less reliable when dealing with neoantigens.

The information used in this step is present in the TCR repertoire and relevant gene expression data received in step S101. The TCR repertoire data is a table containing CDR3 sequences (nucleotide and amino acid), V, D and J alleles and clone or read counts or comparable quantification information, and may be provided by MiXCR for example (Antigen receptor repertoire profiling from RNA-seq data; Dmitriy A Bolotin, Stanislav Poslavsky, Alexey N Davydov, Felix E Frenkel et al.; Nature Biotechnology 35.10 (2017): 908-911). Relevant gene expression may be provided by the FPKM tables discussed above in relation to the gene expression information or, alternatively or in addition, comparable gene expression tables may be provided for use in TCR recognition step S103. These additional gene expression tables include TCR gene specific references. For example, they may contain more different V and J allele sequences and T cell surface marker genes. Additionally, genes relevant for the interaction between T cells and cancer cells are considered, especially checkpoints like CTLA4 and PD1 (and PDL1 on the cancer cell).

Step S103 therefore allows for a prediction of the likelihood of an immune response to each simulated cancer cell being elicited by each candidate neoantigen amino acid sequence. This can then be used in step S104 to select the one or more amino acid sequences for inclusion in the vaccine that maximise the estimated likelihood of the vaccine eliciting an immune response to the cells.

### Optimization: Select the Optimal Neoantigen Vaccine Composition

Let NeoAg={vᵢ}ᵢ₌₀^{N} be a set of neoantigen vaccine element candidates (one of the system inputs). Let C={cⱼ}ⱼ₌₁^{M} be the set of cancer cells simulated by the probabilistic simulation of the cancer cells. We can refer to C as the “cancer digital twin”. Let V⊂NeoAg be an arbitrary subset of NeoAg.

The optimization block of the system aims to find the optimal set V which maximizes the likelihood P (R=+|V, C) of the vaccine inducing an immune response to the simulated population of cells C. Hence, we can formalize the optimisation problem as:

$${max}_{V} P ( R = + \vert V , C ) \qquad ( 1 )$$

This optimisation problem may be addressed in a number of different ways, and two approaches in particular will now be discussed which are based on network flow. As will be understood, other approaches could also be used within the scope of the present invention.

### Embodiment 1

This embodiment is directed towards designing a vaccine which aims at eliciting an immune response meant to kill the maximum number of cancer cells.

From Equation (1), we define:

$${max}_{V} P ( R = + \vert V , C ) := {min}_{V} P ( R = - \vert V , C ) \qquad ( 2 )$$

By modelling the probability of having no immune response in all cells as the joint probability of having immune response in each cell, and assuming conditional independence, we can write:

$${min}_{V} P ( R = - \vert V , C ) = {\prod }_{j = 1}^{M} P ( R = - \vert V , {c}_{j} ) \qquad ( 3 )$$

Let the set of selected vaccine elements V be defined by a set of integer (Boolean) selectors X={xᵢ}ᵢ₌₁^{N}, where

$${x}_{i} = \{ \begin{matrix}1 , & if {v}_{i} ∉ V \\ 0 , & if {v}_{i} ∉ V\end{matrix}$$

The problem of finding the optimal set V is hence the same as finding the optimal set X.

We consider that a vaccine V causes a positive response if at least one of its elements vᵢ induces a positive immune response. That is, the probability of no response P (R=−|V, cⱼ) for a given cell cⱼ is the joint likelihood that all elements fail:

$$P ( R = - \vert V , {c}_{j} ) = {\prod }_{i = 1}^{N} P {( R = - \vert {v}_{i} , {c}_{j} )}^{{x}_{i}} \qquad ( 4 )$$

where xᵢ is the selector of the ith neoantigen and defines its inclusion in the vaccine. In other words, for each neoantigen not selected, the term in the joint likelihood is set to 1, which can be understood as defining that an absent neoantigen cannot induce an immune response.

It follows that:

$$P ( R = - \vert V , C ) = {\prod }_{j = 1}^{M} {\prod }_{i = 1}^{N} P {( R = - \vert {v}_{i} , {c}_{j} )}^{{x}_{i}} \qquad ( 5 )$$

We define the log-probability of not having immune response for a given cell cⱼ by including vaccine element vᵢ in the vaccine as:

$${p}_{ij} : = log P ( R = - \vert {v}_{i} , {c}_{j} ) \qquad ( 6 )$$

We can now rewrite (2) as:

$${min}_{V} {\sum }_{j = 1}^{M} {\sum }_{i = 1}^{N} {p}_{ij} {x}_{i} \qquad ( 7 )$$

For each neoantigen vᵢ we define a cost kᵢ, which can either be constant or a function of its peptide length. (7) is hence constrained by:

$${\sum }_{i = 1}^{N} {k}_{i} {x}_{i} \leq b \qquad ( 8 )$$

where b is a budget which depends on the vaccine platform used.

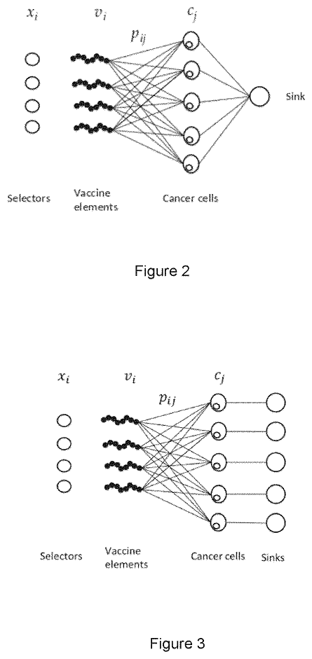

We approach this problem as a type of network flow problem, as illustrated FIG. 2, with one set of nodes corresponding to vaccine elements, one set corresponding to cancer cells, and one sink. The goal is to select the set of vaccine elements such that the likelihood of no response across the whole population of cells is minimized. We can optimize (7) subject to the constraint (8) through integer linear programming (ILP).

### Embodiment 2

Although the same formalization of the problem still holds (see Equation 1), in this embodiment we approach vaccine design by minimizing the probability of no response for the cell which has the highest probability of no response. That is:

$${max}_{V} P ( R = + \vert V , C ) = {min}_{V} {max}_{c ∈ C} \{ P ( R = - \vert V , c ) \} \qquad ( 10 )$$

This approach amounts to designing a vaccine meant to induce at least some response to all cancer cells.

From Equation 4 and Equation 6, we derive:

$${min}_{V} {max}_{1 \leq j \leq M} {\sum }_{i = 1}^{N} {p}_{ij} {x}_{i} \qquad ( 11 )$$

Standard ILP solvers cannot directly solve this minimax problem; however, we use the standard approach of a set of surrogate variables to address this problem. In particular, we define xⱼ^{c} to be the log-likelihood of no response for cell cⱼ. That is:

$${x}_{j}^{c} = {\sum }_{i = 1}^{N} log P ( R = - \vert {v}_{i} {c}_{j} ) {x}_{i} \qquad ( 12 )$$

Further we define:

$$z : = {max}_{1 \leq j \leq M} {x}_{j}^{c} \qquad ( 13 )$$

that is, z is the maximum log-likelihood that any cell does not respond to the vaccine (or, alternatively, the minimum log-likelihood that any cell will respond to the vaccine). Finally, then, our aim is to minimize z:

$${min}_{V} z \qquad ( 14 )$$

Our problem is essentially a min-flow problem with multiple sinks, where each cell is a sink, as shown in FIG. 3; however, our aim is to minimize the flow to each individual sink rather than the flow to all sinks.

### Optimal Vaccine Composition

The outcome of the process is then step S105 in which an optimal vaccine composition has been selected. The composition of this vaccine may be protein-based, in which case the vaccine is formed from amino acid sequences resembling proteins presented on the surface of cancer cells, or may be DNA- or RNA-based, in which case the vaccine is formed from DNA or RNA sequences so as to induce the production of said proteins in a patient's cells.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method of selecting one or more amino acid sequences for inclusion in a neoantigen vaccine from a set of candidate neoantigen amino acid sequences, the method comprising:
retrieving a set of input data related to a patient;
simulating a plurality of cancer cells based on the set of input data, wherein simulating each cancer cell of the plurality of cancer cells comprises predicting a cell surface presentation of said cancer cell of the plurality of cancer cells;
for each candidate neoantigen amino acid sequence of the set of candidate neoantigen amino acid sequences, predicting a likelihood of said candidate neoantigen amino acid sequence eliciting an immune response to the plurality of cancer cells based on the predicted cell surface presentation of each cancer cell of the plurality of cancer cells; and
selecting one or more amino acid sequences of the set of candidate neoantigen amino acid sequences for inclusion in the neoantigen vaccine that maximizes a likelihood of the neoantigen vaccine eliciting an immune response to the plurality of cancer cells based on the predicted likelihood of each candidate neoantigen amino acid sequence eliciting an immune response to the plurality of cancer cells.

2: The computer-implemented method according to claim 1, wherein the set of input data comprises one or more of: an indication of HLA-I alleles of a patient; gene expression information; a set of identified gene variants; binding affinity indicators for each tuple of candidate neoantigen amino acid sequence and HLA-I allele; and presentation indicators for each tuple of candidate neoantigen amino acid sequence and HLA-I allele.

3: The computer-implemented method according to claim 2, wherein simulating the plurality of cancer cells comprises predicting a presence or absence of each identified gene variants of the set of identified gene variants in each cancer cell of the plurality of cancer cells based on a statistical distribution of the set of identified gene variants.

4: The computer-implemented method according to claim 3, wherein simulating the plurality of cancer cells comprises estimating an abundance of one or more proteins synthesized in each cancer cell of the plurality of cancer cells based on the gene expression information and on the set of identified gene variants predicted to be present in each cancer cell.

5: The computer-implemented method according to claim 4, wherein simulating the plurality of cancer cells comprises estimating an abundance of one or more peptides processed in each cancer cell based on the estimated abundance of one or more proteins synthesised in said cancer cell and on a likelihood of each of the one or more proteins being split into the one or more peptides.

6: The computer-implemented method according to claim 5, wherein simulating the plurality of cancer cells comprises simulating a binding of the one or more peptides to HLA molecules to estimate a likelihood of one or more peptide-HLA complexes being present in each cancer cell of the plurality of cancer cells, wherein simulating the binding of the one or more peptides to HLA molecules is based on an abundance of said one or more peptides and on binding affinity indicators for each tuple of candidate neoantigen amino acid sequence and HLA-I allele.

7: The computer-implemented method according to claim 6, wherein simulating the plurality of cancer cells comprises predicting the cell surface presentation of each cancer cell of the plurality of cancer cells based on the likelihood of the one or more peptide-HLA complexes being present within each cancer cell of the plurality of cancer cells and on the presentation indicators for each tuple of candidate neoantigen amino acid sequence and HLA-I allele.

8: The computer-implemented method according to claim 1, wherein predicting the likelihood of each candidate neoantigen amino acid sequence eliciting the immune response to the plurality of cancer cells comprises estimating a likelihood of a patient's immune system including T cells having receptors which bind with the predicted cell surface presentation of each cancer cell.

9: The computer-implemented method according to claim 1, wherein selecting the one or more amino acid sequences of the set of candidate neoantigen amino acid sequences for inclusion in the neoantigen vaccine comprises applying a mathematical optimisation algorithm to minimise a likelihood of the neoantigen vaccine eliciting no immune response to the cancer cells.

10: The computer-implemented method according to claim 9, wherein variables of the mathematical optimisation algorithm comprises:
a binary indicator variable for each candidate neoantigen amino acid sequence of the set of candidate neoantigen amino acid sequences which indicates whether the candidate amino acid is included in the neoantigen vaccine; and
a continuous variable for each cancer cell of the plurality of cancer cells which gives a log likelihood of no immune response being elicited by a candidate neoantigen amino acid sequence of the set of candidate neoantigen amino acid sequences to said cancer cell.

11: The computer-implemented method according to claim 1, wherein selecting the one or more amino acid sequences for inclusion in the neoantigen vaccine comprises applying a mathematical optimisation algorithm to minimise a likelihood of the neoantigen vaccine eliciting no immune response to the each cancer cell for which a likelihood of no immune response being elicited by the neoantigen vaccine is highest.

12: The computer-implemented method according to claim 11, wherein variables of the mathematical optimisation algorithm comprise:
a binary indicator variable for each candidate neoantigen amino acid sequence of the set of candidate neoantigen amino acid sequences which indicates whether the candidate amino acid is included in the neoantigen vaccine;
a continuous variable for each cancer cell of the plurality of cancer cells which gives a log likelihood of no immune response being elicited by a candidate neoantigen amino acid sequence to said cancer cell;
a continuous variable for each cancer cell of the plurality of cancer cells which gives a log likelihood of no immune response being elicited by the neoantigen vaccine comprising a subset of the set of candidate neoantigen amino acid sequences; and
a continuous variable which gives a maximum log-likelihood that any one cancer cell does not respond to the neoantigen vaccine comprising the subset of the set of candidate neoantigen amino acid sequences.

13: The computer-implemented method according to claim 9, wherein the mathematical optimisation algorithm is an integer linear program.

14: The computer-implemented method according to claim 1, wherein the method further comprises assigning a cost to each candidate neoantigen amino acid sequence, and the step of selecting the one or more amino acid sequences for inclusion in the neoantigen vaccine is constrained based on the cost assigned to each candidate neoantigen amino acid sequence, such that the selected one or more amino acid sequences have a total cost below a predetermined threshold budget.

15: A method of creating a vaccine, the method comprising:
selecting one or more amino acid sequences for inclusion in the vaccine from the set of candidate neoantigen amino acid sequences by a method according to claim 1; and
synthesising the one or more selected amino acid sequences or encoding the one or more selected amino acid sequences into a corresponding DNA or RNA sequence and/or incorporating the DNA or RNA sequence into a genome of a bacterial or viral delivery system to create the vaccine.

16: A system for selecting one or more amino acid sequences for inclusion in a vaccine from a set of candidate neoantigen amino acid sequences, the system comprising at least one processor in communication with at least one memory device, the at least one memory device having stored thereon instructions for causing the at least one processor to perform a method according to claim 1.

17: A computer-readable medium having computer executable instructions stored thereon for, when executed by at least one processor, implementing a method according to claim 1.
